(19) United States
(12) Patent Application Publication
(10) Pub. No.: US 2019/0249317 A1
(43) Pub. Date: Aug. 15, 2019

(54) Production of Propanol, Propionaldehyde, and/or Propionic Acid From Carbon Dioxide, Water, and Electrical Energy

(71) Applicant: Siemens Aktiengesellschaft, München (DE)

(72) Inventors: Bernhard Schmid, Erlangen (DE); Günter Schmid, Hemhofen (DE)

(73) Assignee: Siemens Aktiengesellschaft, München (DE)

(21) Appl. No.: 16/333,814

(22) PCT Filed: Aug. 21, 2017
(86) PCT No.: PCT/EP2017/070991

§ 371 Date: Mar. 15, 2019

(30) Foreign Application Priority Data
Sep. 22, 2016 (DE) 10 2016 218 235.8

(51) Int. Cl. C25B 3/04; C25B 1/10; C07C 45/50; C07C 29/16; C07C 51/12; C07C 51/235; C07C 51/285; C25B 11/04; C25B 15/02

(52) U.S. Cl. CPC C25B 3/04; C25B 1/10; C07C 45/50; C07C 29/16; C07C 31/10; C07C 51/235; C07C 51/285; C25B 11/0415; C25B 15/02; C07C 51/12

(57) ABSTRACT

Various embodiments include a method for preparing propanol, propionaldehyde, and/or propionic acid comprising: electrolyzing CO2 to give CO and C2H4; and reacting the CO and C2H4 with H2 to produce propanol and/or propionaldehyde, and/or reacting the CO and C2H4 with H2O to produce propionic acid.

### CROSS-REFERENCE TO RELATED APPLICATIONS

This application is a U.S. National Stage Application of International Application No. PCT/EP2017/070991 filed Aug. 21, 2017, which designates the United States of America, and claims priority to DE Application No. 10 2016 218 235.8 filed Sep. 22, 2016, the contents of which are hereby incorporated by reference in their entirety.

### TECHNICAL FIELD

The present disclosure relates to electrolysis. Various embodiments may include processes for preparing propanol, propionaldehyde, and/or propionic acid, in which CO and C₂H₄ are provided from electrolysis of CO₂, and hydrogen may be provided by the electrolytic means, and the CO and C₂H₄ are reacted with H₂ to give propanol and/or propionaldehyde and/or the CO and C₂H₄ are reacted with H₂O to give propionic acid.

### BACKGROUND

The combustion of fossil fuels currently covers about 80% of global energy demand. These combustion processes emitted about 34 032.7 million metric tons of carbon dioxide (CO₂) globally into the atmosphere in 2011. This release is the simplest way of disposing of large volumes of CO₂ as well (brown coal power plants exceeding 50 000 t per day). Discussion about the adverse effects of the greenhouse gas CO₂ on the climate has led to consideration of reutilization of CO₂. In thermodynamic terms, CO₂ is at a very low level and can therefore be reduced again to usable products only with difficulty.

In nature, CO₂ is converted to carbohydrates by photosynthesis. This process, which is divided up into many component steps over time and spatially at the molecular level, is copiable on the industrial scale only with great difficulty. The more efficient route at present compared to pure photocatalysis is the electrochemical reduction of the CO₂. A mixed form is light-assisted electrolysis or electrically assisted photocatalysis.

As in the case of photosynthesis, in this process, CO₂ is converted to a higher-energy product (such as CO, CH₄, C₂H₄, etc.) with supply of electrical energy (optionally in a photo-assisted manner) which is obtained from renewable energy sources such as wind or sun. The amount of energy required in this reduction corresponds ideally to the combustion energy of the fuel and should only come from renewable sources. However, overproduction of renewable energies is not continuously available, but at present only at periods of strong insolation and wind. However, this state of affairs will further intensify in the near future with the further rollout of renewable energy.

There is currently discussion of the electrification of the chemical industry. This means that chemical commodities or fuels are to be produced preferentially from CO₂ (CO), H₂O with supply of surplus electrical energy, preferably from renewable sources. The aim in the introduction phase of such technology is for the economic value of a substance to be significantly greater than its calorific value.

Electrolysis methods have undergone significant further development in the last few decades. PEM water electrolysis has been optimized toward high current densities, and large electrolyzers having power outputs in the megawatt range are being introduced onto the market. Propionaldehyde and propionic acid are one example of chemical commodities.

Propionaldehyde is typically obtained by hydroformylation of ethene/ethylene:

C₂H₄+CO+H₂→CH₃—CH₂—CHO

With two equivalents, it is also possible to provide propanol, for example with [HCo(phosphine) (CO)₃] as catalyst. Not only ethylene but also H₂ and CO are usually obtained here from fossil sources. Ethylene is obtained, for example, from the steamcracking of naphtha (lst crude oil distillate).

CO in turn can be obtained, for example, by

C+½O₂→CO; C+H₂O→CO+H₂ or coal gasification:

CH₄+H₂O→CO+3H₂. steam reforming of methane:

However, this latter CO:H₂ ratio is unsuitable for hydroformylation. Hydrogen (H₂) can be obtained, for example, by the water-gas shift reaction: CO+H₂O→CO₂+H₂. Alternatively, propionaldehyde and propionic acid can also be prepared by hydration of propene or subsequent oxidation. A further method is propanol oxidation.

However, all these processes for reactant preparation consume fossil energy, lead to by-products and/or do not take place in a suitable phase to conduct hydroformylation, or are very complex. The electrochemical reduction of CO₂ at solid-state electrodes in aqueous electrolyte solutions offers a multitude of possible products that are shown in table 1 below, taken from Y. Hori, Electrochemical CO₂ reduction on metal electrodes, in: C. Vayenas, et al. (eds.), Modern Aspects of Electrochemistry, Springer, New York, 2008, pp. 89-189.

TABLE 1
Faraday efficiencies for carbon dioxide at various metal electrodes

| Electrode | CH₄ | C₂H₄ | C₂H₅OH | C₃H₇OH | CO | HCOO⁻ | H₂ | Total |
|---|---|---|---|---|---|---|---|---|
| Cu | 33.3 | 25.5 | 5.7 | 3.0 | 1.3 | 9.4 | 20.5 | 103.5 |
| Au | 0.0 | 0.0 | 0.0 | 0.0 | 87.1 | 0.7 | 10.2 | 98.0 |
| Ag | 0.0 | 0.0 | 0.0 | 0.0 | 81.5 | 0.8 | 12.4 | 94.6 |
| Zn | 0.0 | 0.0 | 0.0 | 0.0 | 79.4 | 6.1 | 9.9 | 95.4 |
| Pd | 2.9 | 0.0 | 0.0 | 0.0 | 28.3 | 2.8 | 26.2 | 60.2 |
| Ga | 0.0 | 0.0 | 0.0 | 0.0 | 23.2 | 0.0 | 79.0 | 102.0 |
| Pb | 0.0 | 0.0 | 0.0 | 0.0 | 0.0 | 97.4 | 5.0 | 102.4 |
| Hg | 0.0 | 0.0 | 0.0 | 0.0 | 0.0 | 99.5 | 0.0 | 99.5 |
| In | 0.0 | 0.0 | 0.0 | 0.0 | 2.1 | 94.9 | 3.3 | 100.3 |
| Sn | 0.0 | 0.0 | 0.0 | 0.0 | 7.1 | 88.4 | 4.6 | 100.1 |
| Cd | 1.3 | 0.0 | 0.0 | 0.0 | 13.9 | 78.4 | 9.4 | 103.0 |
| Tl | 0.0 | 0.0 | 0.0 | 0.0 | 0.0 | 95.1 | 6.2 | 101.3 |
| Ni | 1.8 | 0.1 | 0.0 | 0.0 | 0.0 | 1.4 | 88.9 | 92.4 |
| Fe | 0.0 | 0.0 | 0.0 | 0.0 | 0.0 | 0.0 | 94.8 | 94.8 |
| Pt | 0.0 | 0.0 | 0.0 | 0.0 | 0.0 | 0.1 | 95.7 | 95.8 |
| Ti | 0.0 | 0.0 | 0.0 | 0.0 | 0.0 | 0.0 | 99.7 | 99.7 |

DE 10 2015 203 245.0 disclosed that high ethylene efficiencies can also be achieved at industrially relevant power densities above 150 mA/cm². However, by-products obtained may still be small amounts of CO and/or considerable amounts of H₂. If pure substances such as ethylene are to be obtained, the process may thus require a further purification stage. There is still a requirement for processes by which basic chemical commodities such as propionaldehyde and propionic acid can be effectively obtained.

### SUMMARY

The present disclosure teaches propanol, propionaldehyde or propionic acid can be prepared effectively when all the commodities required for the propanol, propionaldehyde, or propionic acid synthesis are produced electrochemically. More particularly, a synthesis method for propanol, propionaldehyde or propionic acid with a minimum number of stages and low temperature is described. For slightly elevated temperatures below 100° C., or below 80° C., it is even possible to use the waste heat from the electrolyzer.

For example, some embodiments include a process for preparing propanol, propionaldehyde and/or propionic acid, comprising: electrolysis of CO2 to give CO and C2H4; and reaction of the CO and C2H4 with H2 to give propanol and/or propionaldehyde, and/or reaction of the CO and C2H4 with H2O to give propionic acid.

In some embodiments, H2 is provided by the electrolysis of CO2 and/or CO and/or electrolysis of H2O.

In some embodiments, the electrolysis of CO2 additionally produces an oxygen species at the anode, and the oxygen species is reacted with propanol or propionaldehyde to give propionic acid.

In some embodiments, the oxygen species is oxygen and/or a peroxide.

In some embodiments, C2H4 is prepared from CO2 and/or CO by electrolysis at a copper-containing cathode.

In some embodiments, CO is prepared from CO2 by electrolysis at a cathode comprising a metal selected from the group consisting of Au, Ag and/or Zn.

In some embodiments, the CO and C2H4 are reacted with H2 by a hydroformylation reaction and/or a reaction to prepare propane, and/or wherein CO and C2H4 are reacted with H2O by a hydrocarboxylation reaction.

In some embodiments, waste heat from the electrolysis of CO2 is used in the hydroformylation reaction and/or propane preparation and/or hydroxycarboxylation reaction.

As another example, some embodiments include an apparatus for preparation of propanol, propionaldehyde and/or propionic acid, comprising: at least one first electrolysis unit for the electrolysis of CO2 to give CO and C2H4, which is designed to prepare CO and C2H4 by electrolysis of CO2; and at least one first reactor for reaction of the CO and C2H4 with H2 to give propanol and/or propionaldehyde, and/or for reaction of the CO and C2H4 with H2O to give propionic acid.

In some embodiments, the first electrolysis unit has at least one electrolysis cell having a cathode comprising copper, and has at least one electrolysis cell with a cathode comprising a metal selected from the group consisting of Au, Ag and/or Zn.

As another example, some embodiments include an apparatus for preparation of propanol, propionaldehyde and/or propionic acid, comprising: at least one first electrolysis unit for the electrolysis of CO₂ and/or CO to give C₂H₄, which is designed to prepare C₂H₄ by electrolysis of CO₂ and/or CO; at least one second electrolysis unit for the electrolysis of CO₂ to give CO, which is designed to prepare CO by electrolysis of CO₂; and at least one first reactor for reaction of the CO and C₂H₄ with H₂ to give propionaldehyde and/or propanol, and/or for reaction of the CO and C₂H₄ with H₂O to give propionic acid.

In some embodiments, the first electrolysis unit has a cathode comprising copper, and the second electrolysis unit has a cathode comprising a metal selected from the group consisting of Au, Ag and/or Zn.

In some embodiments, there is at least one third electrolysis unit which is designed to provide H₂ by electrolysis of CO₂ and/or CO and/or electrolysis of H₂O.

In some embodiments, there is a heat conduit designed to supply waste heat from the electrolysis of CO₂ to the first reactor.

In some embodiments, there is a second reactor for conversion of propanol and/or propionaldehyde to propionic acid, which is designed to convert propanol and/or propionaldehyde to propionic acid.

### BRIEF DESCRIPTION OF THE DRAWINGS

The appended drawings are intended to illustrate embodiments of teachings of the present disclosure and to impart further understanding thereof. In connection with the description, they serve to explain concepts and principles herein. Other embodiments and many of the advantages mentioned are apparent with regard to the drawings. The elements of the drawings are not necessarily shown to scale relative to one another. Elements, features, and components that are the same, have the same function and the same effect are each given the same reference numerals in the figures of the drawings unless stated otherwise.

FIGS. 1-5 show, in schematic form, illustrative representations of a possible construction of an electrolysis cell incorporating teachings of the present disclosure;

FIG. 6 shows, in schematic form, one configuration of an electrolysis system for CO₂ reduction without the inventive configuration of the connection between electrolyte supply and gas diffusion electrode;

FIG. 7 shows, in schematic form, one configuration of an electrolysis system for CO₂ reduction with a gas diffusion electrode incorporating teachings of the present disclosure; and

FIG. 8 shows, in schematic form, the progression of a process incorporating teachings of the present disclosure for propionaldehyde preparation.

### DETAILED DESCRIPTION

Some embodiments include a process for preparing propanol, propionaldehyde and/or propionic acid, comprising: electrolysis of CO₂ to give CO and C₂H₄; and reaction of the CO and C₂H₄ with H₂ to give propanol and/or propionaldehyde, and/or reaction of the CO and C₂H₄ with H₂O to give propionic acid. Propanol prepared, if it is not sold directly, optionally after storage, can be converted by oxidation to propionaldehyde and/or propionic acid.

The present process may be a very efficient example of the electrification of the chemical industry. In this case, electrification of the chemical industry means that CO₂, H₂O and power (for the electrolysis), especially electricity surpluses, in some cases from renewable sources, are used to prepare commodities for the chemical industry. The preparation of propanol and/or propionaldehyde is a prime example of this. The CO₂ electrolyzers, owing to side reactions and selectivities, normally give well below 100% gas mixtures that would actually have to be purified for sale and/or further use.

For preparation of propanol and/or propionaldehyde, however, these mixtures, with the possible exception of the removal of excess CO₂, need not be purified or separated since the mixture, in particular embodiments, consists appropriately of ethylene, CO and H₂ for use for a hydroformylation or propanol preparation for instance, or at least only particular proportions of one constituent need be added.

In some embodiments, ethene can be prepared electrolytically either from CO₂ or from CO, which can be obtained from CO₂, such that a sequential progression of the electrolysis is also possible, wherein at least some of the CO prepared at first is converted to C₂H₄, or parallel electrolysis of CO₂ to give ethene and CO can take place. Ethene can also be prepared simultaneously from CO and CO₂, according to the availability of various electrolyzers. Nor is it impossible to use CO from external sources for electrolysis in addition to CO₂, if an excess of CO is present in an external source.

The respective electrolysis of the CO₂ and/or CO is not particularly restricted and can suitably take place with one or more appropriate electrolysis cells or electrolysis units. An electrolysis process is of particular interest since it is a one-step process in which near-worthless, climate-damaging CO₂ or else CO can be used to obtain, with the aid of electricity, energy carriers or chemical commodities.

In some embodiments, CO can be obtained with high selectivity over silver catalysts. Ethene, by contrast, can be formed at copper-based electrodes. Thus, C₂H₄ may be prepared from CO₂ and/or CO in particular embodiments by electrolysis at a copper-containing cathode comprising copper or consisting of copper. In some embodiments, CO is prepared from CO₂ by electrolysis at a cathode comprising a metal or consisting of a metal selected from the group consisting of Au, Ag and/or Zn. In some embodiments, the method uses a silver-containing cathode for CO preparation, which may also, for example, consist of Ag.

In the electrochemical preparation of ethylene from CO₂, as described, it is additionally possible to go via the CO intermediate, as described in detail, for example, in DE 10 2016 200 858.7. The production of ethene and CO can be effected in an electrolysis cell or an electrolysis unit, where it is also possible, for example, to exchange the cathode in an alternating manner in order to prepare different product gases, but can also be effected in two or more electrolysis cells or electrolysis units, where the respective products obtained, such as ethene and CO, which may be present, for example, mixed into water or in moist form, can be mixed in a suitable manner prior to the conversion to propanol, propionaldehyde and/or propionic acid.

Hydrogen can also form, for example, from an electrolysis of water at platinum-containing cathodes, but also often forms as a by-product in an electrolysis of CO₂, as apparent from table 1 above, and so it may be the case that no separate H₂ electrolyzer is required either. For example, the ethylene and CO prepared electrolytically, or as a result of the competing reaction with H₂O, may thus comprise H₂. CO prepared at silver electrodes, for example, likewise contains small to considerable amounts of H₂.

In some embodiments, H₂ is thus prepared by the electrolysis of CO₂ and/or electrolysis of H₂O. The electrolysis of water here, like the electrolysis of CO₂, is not particularly restricted and may include customary electrolyzers of water. Illustrative reactions in the electrolysis for preparation of CO, ethene and hydrogen are as follows:

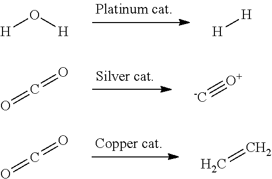

The cathode reactions required for this purpose are, for example:

2H₂O+2e⁻→H₂+2OH⁻ Hydrogen:

CO₂+2e⁻+H₂O→CO+2OH⁻ Carbon monoxide:

2CO₂+12e⁻+8H₂O→C₂H₄+12OH⁻ Ethylene:

These cathode reactions can be combined virtually as desired with various anode reactions.

Examples here include:

2Cl⁻→Cl₂+2e⁻ chloralkali production:

It is possible here, as described in DE 10 2015 212 504.1, for considerable amounts of valuable NaHCO₃ to be obtained, which can be processed further to soda.

2H₂O-4e⁻→O₂+4H⁺ oxygen production:

2H₂O-2e⁻→H₂O₂+2H⁺ hydrogen production:

2SO₄²⁻2e⁻→S₂O₈²⁻ peroxodisulfate production:

Oxygen compounds produced at the anode, such as O₂, peroxides such as peroxodisulfate can be used for oxidation of the propanol and/or propionaldehyde to propionic acid, which once again underlines the synergy of the overall process. In this case, it is also possible to use waste heat from the electrolysis or the electrolysis units for oxidation of the propanol and/or propanol/propionaldehyde, e.g. propanal, for example in the presence of cobalt or manganese ions, for example at 40-50° C., in particular embodiments. This oxidation can be effected in a second reactor of the apparatuses of the invention. The propanol and/or propionaldehyde can thus be used to prepare propionic acid per se via incorporation of the anode reaction.

In some embodiments, the electrolysis of CO₂ thus additionally produces an oxygen species at the anode, and the oxygen species can be reacted with propanol and/or propionaldehyde to give propionic acid. In some embodiments, the oxygen species is oxygen and/or a peroxide such as hydrogen peroxide or peroxodisulfate.

As stated above, the electrolysis processes and the electrolysis cells or electrolysis units/electrolysis systems/electrolyzers used for the purpose are not particularly restricted. The individual electrolyzers may be of different configuration. Conceivable hydrogen electrolyzers are, for example, those with polymer electrolyte membrane, and/or alkaline or chloralkali electrolyzers.

The electrolytes of the CO₂ electrolyzers, in particular embodiments, contain alkali metal cations, more preferably Na⁺ and/or K⁺. Preferred anions are, for example, carbonate, hydrogencarbonate, sulfate, hydrogensulfate and/or phosphates. These can be chosen suitably according to the anode reaction. The electrolytes may also contain or consist of additions such as ionic liquids.

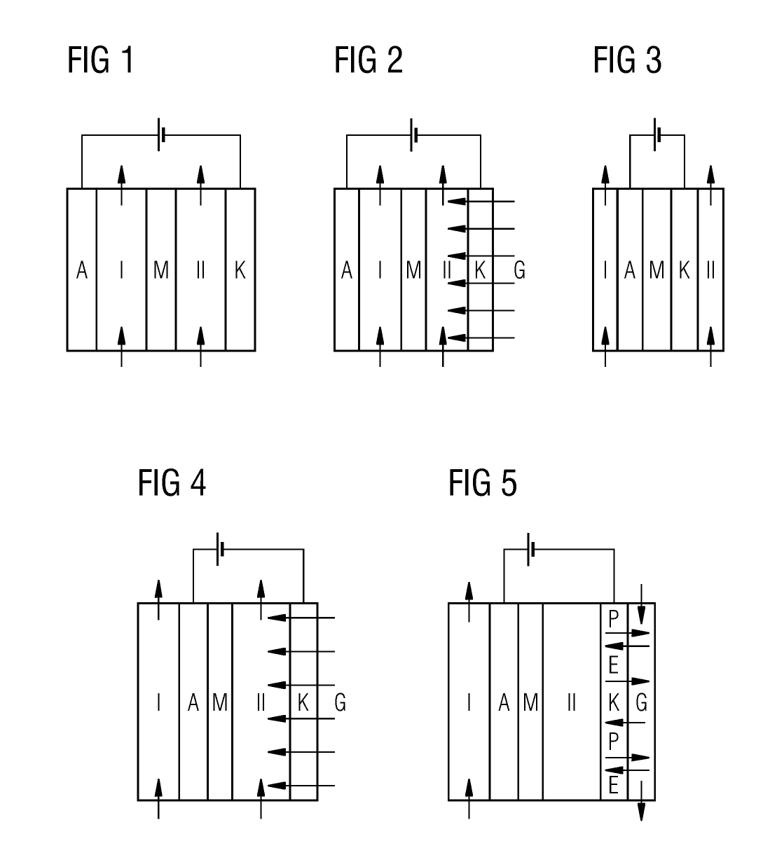

FIGS. 1-5 show illustrative diagrams of a possible construction of an electrolysis cell, for example carbon dioxide reduction or carbon monoxide reduction, which can be employed in the process and the apparatuses described herein, wherein the anodes and cathode regions thereof may be combined with one another as desired.

In some embodiments, the electrolysis cell of an electrolysis unit that can be employed in the process comprises at least one anode and one cathode, one of which may take the form of a gas diffusion electrode, for example, and a cell space designed to be filled with an electrolyte and into which the anode and cathode have been at least partly introduced. In some embodiments, both the anode and cathode take the form of a gas diffusion electrode. In particular embodiments, the anode takes the form of a gas diffusion electrode. In particular embodiments, the cathode takes the form of a gas diffusion electrode. In particular embodiments, in carbon dioxide electrolysis or carbon monoxide electrolysis, carbon dioxide and/or carbon monoxide is electrolytically converted at the cathode, i.e. the cathode is in such a form that it can convert carbon dioxide and/or carbon monoxide, for example of a copper- and/or silver-containing gas diffusion electrode.

The electrolysis cells used correspond, for example, to those shown in schematic form in FIGS. 1 to 5; the figures show cells with a membrane M which may also be absent in the apparatuses of the invention, but is employed in particular embodiments, and which can separate an anode space I and a cathode space II. If a membrane is present, it is not particularly restricted and is matched, for example, to the electrolysis, for example to the electrolyte and/or the anode reaction and/or cathode reaction.

In some embodiments, the electrochemical reduction, for example of CO₂, takes place in an electrolysis cell that typically consists of an anode space and a cathode space. FIGS. 1 to 5 show examples of a possible cell arrangement. A gas diffusion electrode may be used for any of these cell arrangements, for example as cathode.

By way of example, the cathode space II in FIGS. 1 and 2 is configured such that a catholyte is supplied from the bottom and then leaves the cathode space II at the top. In some embodiments, the catholyte can also be supplied from the top, as in the case of falling-film electrodes for example. At the anode A, which is electrically connected to the cathode K by means of a power source for provision of the potential for the electrolysis, the oxidation of a substance which is supplied from the bottom together with an anolyte, for example, takes place in the anode space I, and the anolyte then leaves the anode space together with the product of the oxidation.

In the 3-chamber construction shown in FIGS. 1 and 2, a reaction gas, for example carbon dioxide and/or carbon monoxide, can be conveyed through and/or along a cathode, for example a gas diffusion electrode, here by way of example the cathode K, into the cathode space II for reduction, by way of example as in FIG. 1 (in backflow operation, if the cathode takes the form of a gas diffusion electrode) or in through-flow operation in FIG. 2 (with a gas diffusion electrode). In some embodiments, there is a porous anode A.

In FIGS. 1 and 2, the spaces I and II are separated by a membrane M. By contrast, in the PEM (proton or ion exchange membrane) construction of FIG. 3, the cathode K, for example a gas diffusion electrode, and an anode A, for example a porous anode, are directly adjacent to the membrane M, which separates the anode space I from the cathode space II. The construction in FIG. 4 corresponds to a mixed form of the construction from FIG. 2 and the construction from FIG. 3, with provision of a construction with the gas diffusion electrode and gas supply G in through-flow operation on the catholyte side, as shown in FIG. 2, whereas a construction as in FIG. 3 is provided on the anolyte side. Of course, mixed forms or other configurations of the electrode spaces shown by way of example are also conceivable.

Also conceivable are embodiments without a membrane. In some embodiments, the electrolyte on the cathode side and the electrolyte on the anode side may thus be identical, and the electrolysis cell/electrolysis unit may not need a membrane, although a membrane may be present for gas separation. However, it is thus not impossible that the electrolysis cell in such embodiments has a membrane, but this is associated with additional complexity with regard to the membrane and also the potential applied. Catholyte and anolyte may also optionally be mixed again outside the electrolysis cell.

FIG. 5 corresponds to the construction of FIG. 4, where the gas supply G here takes place in backflow operation and the passage of reactant and product E and P are shown.

FIGS. 1 to 5 are schematic diagrams. The electrolysis cells from FIGS. 1 to 5 may also be combined to form mixed variants. For example, the anode space may be designed as a PEM half-cell, as in FIG. 3, while the cathode space consists of a half-cell including a certain electrolyte volume between membrane and electrode, as shown in FIG. 1. The membrane may also be in multilayer form, such that separate feeds of anolyte and catholyte are enabled. Separation effects in the case of aqueous electrolytes are achieved, for example, via the hydrophobicity of interlayers.

Conductivity can nevertheless be assured when conductive groups are integrated into such separation layers. The membrane may be an ion-conducting membrane, or a separator, which brings about solely mechanical separation, e.g. gas separation, and is permeable to cations and anions.

The use of a gas diffusion electrode makes it possible to construct a three-phase electrode. For example, a gas can be guided from the back to the electrically active front side of the electrode in order to conduct an electrochemical reaction there. In particular embodiments, the gas diffusion electrode may also be operated merely with backflow, meaning that a gas such as CO₂ and/or CO is guided past the back side of the gas diffusion electrode in relation to the electrolyte, in which case the gas can penetrate through the pores of the gas diffusion electrode and the product can be removed at the back. For example, the gas flow in the case of backflow is the reverse of the flow of the electrolyte, in order that liquid that has been forced through, such as electrolyte, can be transported away.

The gas diffusion electrodes, for example for high current densities, can thus work in two fundamentally different modes of operation:

a. a gas such as CO₂ and/or CO is forced through the cathode.

b. a gas such as CO₂ and/or CO flows past behind the cathode.

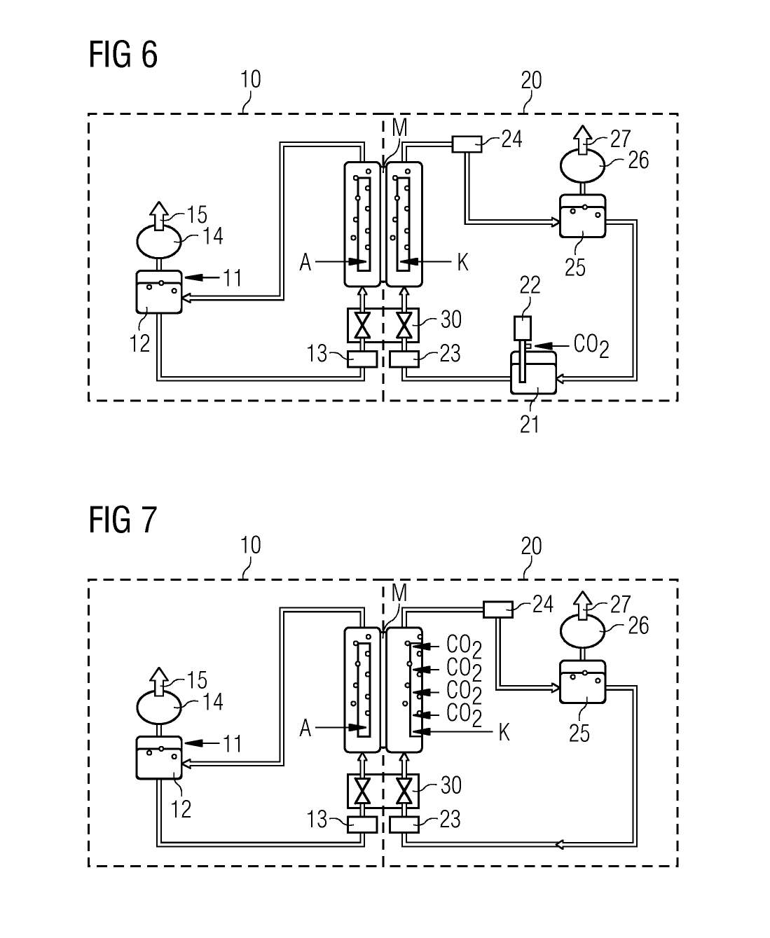

An illustrative electrolysis unit for CO₂ electrolysis is shown in FIG. 6 but is also analogously conceivable for a CO electrolysis for example.

An electrolysis unit is shown, in which carbon dioxide is reduced on the cathode side and water is oxidized on the anode A side. On the anode side, it would alternatively be possible, for example, for a reaction of chloride to give chlorine, bromide to give bromine, sulfate to give peroxodisulfate (with or without evolution of gas), etc. to take place. An example of a suitable anode A is platinum, and an example of a suitable cathode K is copper. The two electrode spaces of the electrolysis cell are separated in the figure by a membrane M, for example of Nafion®. The incorporation of the cell into a system with anolyte circuit 10 and catholyte circuit 20 is shown in the figure.

On the anode side, water with electrolyte additions is fed into an electrolyte reservoir vessel 12 via an inlet 11. However, it is not impossible that water is supplied additionally or instead of the inlet 11 at another point in the anolyte circuit 10, since, according to FIG. 6, the electrolyte reservoir vessel 12 can also be used for gas separation. Water/electrolyte is pumped out of the electrolyte reservoir vessel 12 by means of the pump 13 into the anode space, where it is oxidized. The product is then pumped back into the electrolyte reservoir vessel 12, where it can be removed into the product gas vessel 26. The product gas can be withdrawn from the product gas vessel 26 via a product gas outlet 27. The product gas can of course also be removed elsewhere. The result is thus an anolyte circuit 10 since the electrolyte is being circulated on the anode side.

On the cathode side, in the catholyte circuit 20, carbon dioxide is introduced via a CO₂ inlet 22 into an electrolyte reservoir vessel 21, where it is physically dissolved for example. By means of a pump 23, this solution is introduced into the cathode space, where the carbon dioxide is reduced at the cathode K, for example to CO at a silver cathode. An optional further pump 24 then pumps the solution containing CO which is obtained at the cathode K further to a vessel for gas separation 25, where the product gas containing CO can be removed into a product gas vessel 26. The product gas can be removed from the product gas vessel 26 via a product gas outlet 27. The electrolyte is in turn pumped out of the vessel for gas separation back to the electrolyte reservoir vessel 21, where carbon dioxide can be added again. Here too, merely an illustrative arrangement of a catholyte circuit 20 is specified, where the individual apparatus components of the catholyte circuit 20 may also be arranged differently, for example in that the gas separation is already effected in the cathode space. Preferably, the gas separation and gas saturation are effected separately; in other words, the electrolyte is saturated with CO₂ in one of the vessels and then pumped through the cathode space as a solution without gas bubbles. In that case, the gas that exits from the cathode space consists of CO in a predominant proportion, since CO₂ itself remains dissolved since it has been consumed and hence the concentration in the electrolyte is somewhat lower.

Electrolysis in FIG. 6 is effected by addition of power via a power source (not shown). In order to be able to supply the electrolysis unit with the water and the CO₂ dissolved in the electrolyte with variable pressure over time, valves 30 may be introduced into the anolyte circuit 10 and catholyte circuit 20, and these may be controlled with a control unit (not shown) and hence control the supply of anolyte and catholyte to the anode and cathode, which enables supply with variable pressure and purging of product gas out of the respective electrode cells.

In the figure, the valves 30 are shown upstream of the inlet into the electrolysis cell, but may also, for example, be provided downstream of the outlet from the electrolysis cell and/or at other points in the anolyte circuit 10 or catholyte circuit 20. It is also possible, for example, for a valve 30 to be present upstream of the inlet into the electrolysis cell in the anolyte circuit, whereas the valve in the catholyte circuit 20 is beyond the electrolysis cell, or vice versa.

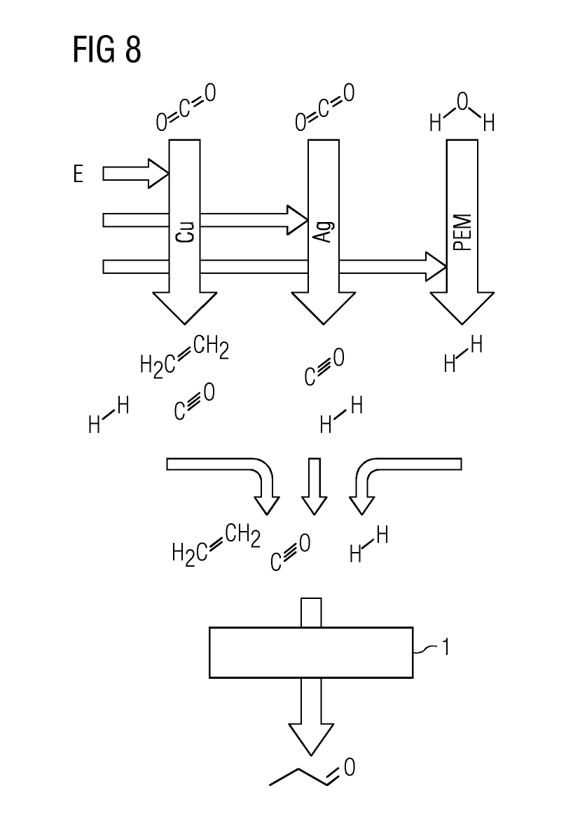

A further electrolysis unit for CO₂ shown by way of example in FIG. 7 corresponds to the electrolysis unit in FIG. 6, where the cathode here takes the form of a through-flow gas diffusion electrode. This electrolysis unit too is employable analogously for CO. As a result of the electrolysis or electrolyses, a gas mixture suitable as starting gas for the preparation of propanol, propionaldehyde and/or propionic acid or esters thereof can be obtained.

Since the present carbon dioxide electrolysis is a gas-to-gas electrolysis, the product gases typically also contain CO₂, which can easily be removed by a gas scrubbing operation (pressurized water scrubbing, absorption scrubbing). Thus, in particular embodiments, the product gas from the electrolysis, especially the CO₂ electrolysis, before being supplied to the conversion to propanol, propionaldehyde and/or propionic acid, is cleaned by a gas scrubbing operation which is not particularly restricted. The apparatuses of the invention thus comprise, in particular embodiments, one or more gas scrubbers provided between the electrolysis units, especially the first and any second (CO₂) electrolysis units, and the first reactor for conversion.

In some embodiments, the reaction of the CO and C₂H₄ with H₂ to give propanol, propionaldehyde and/or the reaction of the CO and C₂H₄ with H₂O to give propionic acid is not particularly restricted and can be effected by known methods. In some embodiments, the respective reactions are effected in water, which may already be used as solvent in the electrolysis, and so there is no need for any separation of CO, C₂H₄ and/or H₂ from the water prior to the reaction. Like the respective reaction, the first reactor used for the purpose, especially in the apparatuses of the invention, is not particularly restricted.

In some embodiments, the CO and C₂H₄ are reacted with H₂ by a hydroformylation reaction. In particular embodiments, the CO and C₂H₄ are reacted with appropriate equivalents of H₂ to give propanol, for example with [HCo(phosphine) (CO)₃] as catalyst. In particular embodiments, the CO and C₂H₄ are reacted with H₂O by a hydrocarboxylation reaction, for example using nickel carbonyl as catalyst.

Hydroformylation is an ideal application for an ethene electrolyzer with the experimentally attained product gas composition since the “by-products” are also required. Since ethene, CO and H₂ are required in equimolar amounts, in particular embodiments, these are added, for example, in addition to the product gas stream for the ethene electrolyzer. For this purpose, for example, it is possible to use a CO₂ and/or CO electrolyzer and optionally a water electrolyzer. According to the design and catalyst selectivity, the starting mixture for the hydroformylation may come from one electrolyzer or the combination of two or even three electrolyzers.

By combination of an ethene, CO and water electrolyzer, it is possible to produce gas mixtures that are suitable in principle as feed for hydroformylation, and also for propanol production. The coupling of electrolysis and hydroformylation for production of propionaldehyde or else the coupling of electrolysis and propanol production are also of particular economic interest because the individual gases need not be subjected to further workup.

In principle, coupling with all variants of hydroformylation or of propanol preparation is possible.

In some embodiments, the hydroformylation is effected by a rhodium-catalyzed hydroformylation, for example in biphasic mode. It may be conducted in aqueous solution, with no requirement for drying of the product gas stream. In particular embodiments, the gas is saturated with water, and so aqueous electrolytes may be used in the respective electrolysis. In the biphasic process, the Rh complex that functions as catalyst is dissolved in an aqueous phase. Since the aldehydes produced do not mix completely with water, the product separates out at least partly as second phase.

Moreover, the reactants are gaseous. Therefore, this process can be conducted continuously. This makes it particularly suitable for coupling to electrolysis systems since electrolyzers typically work continuously. In the proposed coupling, accordingly, there is also no need for any complex intermediate storage of the reactor gases.

The reaction temperature for the hydroformylation to give propionaldehyde is usually in the range of 60-80° C. This means that this reaction can be conducted, for example, at least partly or else completely with the waste heat from the electrolyzers. This temperature profile even enables, in particular embodiments, the distillative removal of the propionaldehyde (boiling point 49° C.). The waste heat from the electrolyzer may thus be sufficient to operate the hydroformylation reactor and to remove the propionaldehyde by distillation. Analogous considerations may be made for the propanol production, in which at least two equivalents of H₂ are correspondingly required.

A similar case is that of hydrocarboxylation of ethene, it being possible here to directly use, for example, water from an electrolyzer in which ethene and/or CO are dissolved, especially with nickel carbonyl. Here too, coupling with continuous electrolysis units is possible. This reaction can also be conducted, for example, at least partly or completely with the waste heat from the electrolyzers.

In some embodiments, waste heat from the electrolysis of CO₂ is thus used in the hydroformylation reaction, propanol production and/or hydrocarboxylation reaction. Waste heat from the electrolyzer(s) can be used for said methods of conversion to propanol, propionaldehyde and/or propionic acid, for example at slightly elevated temperatures below 100° C., preferably below 90° C., further preferably below 80° C.

In some embodiments, rather than a reaction with water to give propionic acid, a reaction with alcohol R—OH to give propionic esters is also possible, where R here may be a substituted or unsubstituted, for example unsubstituted, organic radical having 1 to 20, for example 1 to 6, 1 to 4 or 1 to 2, carbon atoms, for example a substituted or unsubstituted, for example unsubstituted, alkyl, aryl, alkylaryl or arylalkyl radical having 1 to 20, for example 1 to 6, 1 to 4 or 1 to 2, carbon atoms. The substituents are not restricted, provided that they do not interfere and are not converted in the reaction, and may, for example, be halogen radicals, —OH, etc.

Thus, a process for preparing esters of propionic acid is also described, comprising:
electrolysis of CO₂ to give CO and C₂H₄; and
reaction of the CO and C₂H₄ with ROH to give propionic esters, where ROH is as defined above. In this case too, the individual embodiments with regard to the electrolysis and reaction find use analogously, and also with regard to a corresponding apparatus.

Also described are an apparatus for preparation of propionic esters, comprising:
at least one first electrolysis unit for the electrolysis of CO₂ to give CO and C₂H₄, which is designed to prepare CO and C₂H₄ by electrolysis of CO₂; and
at least one first reactor for reaction of the CO and C₂H₄ with ROH to give propionic esters; and
   an apparatus for preparation of propionic esters, comprising:
at least one first electrolysis unit for the electrolysis of CO₂ and/or CO to give C₂H₄, which is designed to prepare C₂H₄ by electrolysis of CO₂ and/or CO;
at least one second electrolysis unit for the electrolysis of CO₂ to give CO, which is designed to prepare CO by electrolysis of CO₂; and
at least one first reactor for reaction of the CO and C₂H₄ with ROH to give propionic esters, where ROH is again as defined above.

In some embodiments, there is an apparatus for preparation of propanol, propionaldehyde and/or propionic acid, comprising:

at least one first electrolysis unit for the electrolysis of CO₂ to give CO and C₂H₄, which is designed to prepare CO and C₂H₄ by electrolysis of CO₂; and

at least one first reactor for reaction of the CO and C₂H₄ with H₂ to give propionaldehyde and/or propanol, and/or for reaction of the CO and C₂H₄ with H₂O to give propionic acid.

In such a construction, it is possible that, for example, one electrolysis cell is operated with varying cathodes in order to produce different product gases, although this requires intermediate storage of product gases, or it is possible to operate an electrolysis unit with multiple electrolysis cells which can work in parallel, for example, in which case, for example, it is also possible to adjust the number of different electrolysis cells in order to obtain a virtually ideal reactant gas mixture by mixing the products from the electrolysis cells, and this can then be supplied to a hydroformylation reaction or hydrocarboxylation reaction. Sequential electrolysis cells for preparation of ethene from CO₂ via the CO intermediate are also possible.

In some embodiments, the first electrolysis unit has at least one electrolysis cell with a cathode comprising or consisting of copper and has at least one electrolysis cell with a cathode comprising or consisting of a metal selected from the group consisting of Au, Ag and/or Zn. In this way, it is possible, for example in a parallel manner, to efficiently prepare ethene and CO and possibly also H₂.

In some embodiments, there is an apparatus for preparation of propanol, propionaldehyde and/or propionic acid, comprising:

at least one first electrolysis unit for the electrolysis of CO₂ to give C₂H₄, which is designed to prepare C₂H₄ by electrolysis of CO₂;

at least one second electrolysis unit for the electrolysis of CO₂ to give CO, which is designed to prepare CO by electrolysis of CO₂; and

at least one first reactor for reaction of the CO and C₂H₄ with H₂ to give propionaldehyde and/or propanol, and/or for reaction of the CO and C₂H₄ with H₂O to give propionic acid.

In some embodiments, there is a subsequent oxidation of the propanol and/or propionaldehyde with oxygen produced at the anode to give propionic acid in a suitable reactor. It is possible here to prepare CO and ethylene-ethylene from CO as well, for example—in parallel in different electrolysis units, which can increase the efficiency of the process of the invention in the case of performance in such an apparatus.

In some embodiments, the first electrolysis unit has a cathode comprising or consisting of copper, and the second electrolysis unit has a cathode comprising or consisting of a metal selected from the group consisting of Au, Ag and/or Zn. In this way, it is again possible to efficiently prepare, for example in parallel, ethene and CO and possibly also H₂. A suitable substrate for preparation of ethylene and hydrogen is CO₂, but also CO.

In some embodiments, the apparatuses further include at least one third electrolysis unit designed to provide H₂ by electrolysis of CO₂ and/or CO and/or electrolysis of H₂O. With a combination of two or especially three electrolysis units, it is possible to prepare, with high efficiency, a suitable mixture for hydroformylation and/or propanol production.

In the first reactor, in the apparatuses, it is possible to conduct a hydroformylation reaction, a reaction for preparation of propanol, or a hydrocarboxylation reaction, where the first reactor here is not particularly restricted.

In some embodiments, there is at least one heat conduit designed to supply waste heat from the electrolysis of CO₂ to the first reactor. A corresponding heat conduit can also provide waste heat from other electrolysis units, for example a CO and/or H₂O electrolysis. Such a heat conduit may also be provided for supply of waste heat to a second reactor for conversion of propanol and/or propionaldehyde to propionic acid. The heat conduits here are not particularly restricted. Rather than heat conduits, it is also possible to provide direct contact between a respective (first, second and/or third) electrolysis apparatus and a respective (first and/or second) reactor.

In some embodiments, the apparatuses comprise a second reactor for conversion of propanol and/or propionaldehyde to propionic acid, which is designed to convert propanol and/or propionaldehyde to propionic acid. This reactor too is not particularly restricted, provided that it permits the corresponding conversion, for example oxidation.

The apparatuses described herein can be used to execute the processes described herein. In this respect, the respective electrolysis units and reactors correspond, for example, to those mentioned in connection with the process. In addition, the apparatus may comprise further constituents present in an electrolysis system or electrolysis unit, as well as the power source for the electrolysis, various cooling and/or heating units, etc., and also constituents of a reactor such as cooling and/or heating units, and connections between the electrolysis units and reactors, for example in the form of pipes etc. These further constituents of the apparatus, for example of an electrolysis system, are not subject to any further restriction and may be provided in a suitable manner.

The above embodiments, configurations, and developments can, if viable, be combined with one another as desired. Further possible configurations, developments and implementations of the teachings herein also include combinations that have not been explicitly specified of features of the invention that are described above or hereinafter with regard to the working examples. More particularly, the person skilled in the art will also add individual aspects as improvements or additions to the respective basic form.

The teachings herein are described hereinafter with reference to some illustrative embodiments, but these do not restrict the scope of the disclosure.

### Examples

An example process is shown in schematic form by way of example in FIG. 8 for a hydroformylation. In this case, energy E, for example from renewable energy sources and/or surplus power, is used in the respective electrolysis steps for Cu-catalyzed preparation of C₂H₄, optionally with CO and H₂ as by-products, from CO₂, for Ag-catalyzed preparation of CO, optionally with H₂ as by-product, from CO₂, and for PEM electrolysis of water to give H₂. These reactants are then mixed and converted to propionaldehyde in the hydroformylation 1. The process described provides a highly integrated, energy-optimized process for preparing propionaldehyde and propionic acid without fossil raw materials and high-temperature processes.

Since all the components required can be produced electrochemically from CO₂, the process is independent of fossil carbon sources. The energy input is additionally concentrated mainly in the electrochemical steps, which distinctly increases energy efficiency. It is not least the case that a versatile C3 unit is obtained from the energy-free and worthless C1 unit CO₂. An ethylene/CO/H₂ mixture would not be saleable without costly separation, whereas the C3 derivatives from the combined process constitute a directly saleable product.

What is claimed is:

1. A method for preparing propanol, propionaldehyde, and/or propionic acid, the method comprising:
electrolyzing CO2 to give CO and C2H4; and
reacting the CO and C2H4 with H2 to produce propanol and/or propionaldehyde, and/or reacting the CO and C2H4 with H2O to produce propionic acid.

2. The process as claimed in claim 1, further comprising generating H2 by electrolysis of CO2 and/or CO and/or electrolysis of H2O.

3. The process as claimed in claim 1, further comprising:
producing an additional oxygen species during the electrolysis of CO2 at the anode; and
reacting the oxygen species with propanol or propionaldehyde to give propionic acid.

4. The process as claimed in claim 3, wherein the oxygen species comprises oxygen and/or a peroxide.

5. The process as claimed in claim 1, further comprising preparing C2H4 from CO2 and/or CO by electrolysis at a copper-containing cathode.

6. The process as claimed in claim 1, further comprising preparing CO from CO2 by electrolysis at a cathode comprising a metal selected from the group consisting of: Au, Ag, and Zn.

7. The process as claimed in claim 1, further comprising:
reacting CO and C2H4 with H2 using a hydroformylation reaction and/or a reaction to prepare propane, and/or
reacting CO and C2H4 with H2O using a hydrocarboxylation reaction.

8. The process as claimed in claim 7, further comprising using waste heat from the electrolysis of CO2 in the hydroformylation reaction and/or propane preparation and/or hydroxycarboxylation reaction.

9. An apparatus for preparation of propanol, propionaldehyde, and/or propionic acid, the apparatus comprising:
a first electrolysis unit for the electrolysis of CO2 to give CO and C2H4; and
a first reactor for reaction of CO and C2H4 with H2 to give propanol and/or propionaldehyde, and/or for reaction of CO and C2H4 with H2O to give propionic acid.

10. The apparatus as claimed in claim 9, wherein the first electrolysis unit comprises a cathode comprising copper and a cathode comprising a metal selected from the group consisting of: Au, Ag, and Zn.

11. An apparatus for preparation of propanol, propionaldehyde, and/or propionic acid, the apparatus comprising:
a first electrolysis unit for the electrolysis of CO₂ and/or CO to give C₂H₄;
a second electrolysis unit for the electrolysis of CO₂ to give CO; and
a first reactor for reaction of CO and C₂H₄ with H₂ to give propionaldehyde and/or propanol, and/or for reaction of the CO and C₂H₄ with H₂O to give propionic acid.

12. The apparatus as claimed in claim 11, wherein:
the first electrolysis unit includes a cathode comprising copper; and
the second electrolysis unit includes a cathode comprising a metal selected from the group consisting of: Au, Ag, and Zn.

13. The apparatus as claimed in claim 9, further comprising a third electrolysis unit to provide H₂ by electrolysis of CO₂ and/or CO and/or electrolysis of H₂O.

14. The apparatus as claimed in claim 9, further comprising a heat conduit to channel waste heat from the electrolysis of CO₂ to the first reactor.

15. The apparatus as claimed in claim 9, further comprising a second reactor for conversion of propanol and/or propionaldehyde to propionic acid.
